(19) United States
(12) Patent Application Publication
(10) Pub. No.: US 2024/0239742 A1
(43) Pub. Date: Jul. 18, 2024

(54) SYNTHESIS OF VITAMIN D3 FROM CHOLESTEROL EXTRACTED FROM FISH OIL

(71) Applicants: Jhesua Valencia Ocampo, Barranquilla (CO); Wilson de Jesús Martinez Gutierrez, Barranquilla (CO); Wendy Katherine Valdez Cantillo, Barranquilla (CO)

(72) Inventors: Jhesua Valencia Ocampo, Barranquilla (CO); Wilson de Jesús Martinez Gutierrez, Barranquilla (CO); Wendy Katherine Valdez Cantillo, Barranquilla (CO)

(21) Appl. No.: 18/399,352

(22) Filed: Dec. 28, 2023

Related U.S. Application Data
(60) Provisional application No. 63/436,038, filed on Dec. 29, 2022.

(51) Int. Cl. C07C 401/00

(52) U.S. Cl. CPC C07C 401/00

(57) ABSTRACT

The present invention provides synthetic methods and preparation of cholecalciferol (vitamin D₃) from cholesterol, which is a by-product of the Omega-3 oil production process. According to the invention, dehydro-cholesterol is prepared and subjected to UV radiation (254 nm). Accordingly, two new synthetic alternatives are provided; wherein the first consists of processes of oxidation-reduction of cholesterol to form dehydro-cholesterol and subsequent UV radiation; which route involves six steps (see scheme 1). The second transformation involves the generation of vitamin D₃ by means of a bromination method and a photocatalytic reaction using UV radiation; which alternative requires four steps (see scheme 2).

This application claims the priority benefit under 35 U.S.C. section 119 of U.S. Provisional Patent Application No. 63/436,038 entitled “Synthesis Of Vitamin D₃ From Cholesterol Extracted From Fish Oil” filed on Dec. 29, 2022.

The invention relates to the field of the fine chemical industry, in particular to production processes for vitamin D₃ in compliance with pharmacopoeia standards. The invention further belongs to the field of organic synthesis, and in particular relates to a preparation method of 7-dehydrocholesterol and vitamin D₃. The present invention further relates to an improved synthesis of vitamin D₃. The present invention additionally relates to the field of molecular synthesis and, more particularly, to the photochemical synthesis of vitamin D from precursor molecules.

### BACKGROUND OF THE INVENTION

Vitamin D refers to a group of fat-soluble secosteroids responsible for enhancing intestinal absorption of calcium, iron, magnesium, phosphate and zinc. In humans, the most important compounds in this group are vitamin D₃ (cholecalciferol) and vitamin D₂ (ergocalciferol). Vitamin D is naturally present in certain foods, added to others, and available as a dietary supplement.

Vitamin D₃, also known as cholecalciferol, is a colorless crystal with a melting point of 84-85° C. It is a fat-soluble substance, insoluble in water, but soluble in solvents such as alcohol, ether, acetone, chloroform, and vegetable oil. Vitamin D₃ is easily oxidized and deactivated in humid air, but it can withstand high temperature and oxidation in neutral and alkaline solutions. Therefore, it should be stored in nitrogen, light-free and acid-free cold environments.

Vitamins are found in human and most animal tissues. Human skin contains its precursor dehydrocholesterol, which can be transformed into vitamins under sunlight or ultraviolet radiation. Foods such as cod liver oil, liver, roe, butter, milk, and egg yolk are also rich in vitamins.

Vitamin D₃ is an essential substance for normal growth and reproduction of humans and animals, and it is also an active form of vitamin D with the highest biological metabolism rate. When vitamin D₃ is converted into activated vitamin D₃ in the liver and kidney in turn, it can regulate calcium and phosphorus metabolism, regulate cell differentiation and regulate the immune system. When the human body lacks vitamin D₃, the ability to absorb calcium and phosphorus is reduced. Calcium and phosphorus cannot be deposited in bone tissue, and even dissolve bone salt, hindering the consumption of bone. Children will suffer from canine rickets if they are deficient, while adults will suffer from osteomalacia. Vitamin D₃ is often used as a drug and food additive. Due to its high-efficiency and low-toxic therapeutic index, activated vitamin D₃ and its homologues can be used to treat secondary parathyroid dysfunction, osteoporosis, psoriasis, Medicines for diseases such as kidney failure.

Cholecalciferol is made in the skin following UVB light exposure. It is converted in the liver to calcifediol (25-hydroxyvitamin D) which is then converted in the kidney to calcitriol (1,25-dihydroxyvitamin D). One of its actions is to increase calcium uptake by the intestines. It is found in food such as some fish, beef liver, eggs, and cheese. Plants, cow milk, fruit juice, yogurt, and margarine also may have cholecalciferol added to them in some countries, including the United States.

Cholecalciferol can be taken as an oral dietary supplement to prevent vitamin D deficiency or as a medication to treat associated diseases, including rickets. It is also used for familial hypophosphatemia, hypoparathyroidism that is causing low blood calcium, and Fanconi syndrome. Vitamin-D supplements may not be effective in people with severe kidney disease. Excessive doses in humans can result in vomiting, constipation, weakness, and confusion. Other risks include kidney stones. Doses greater than 40,000 IU (1,000 μg) per day are generally required before high blood calcium occurs. Normal doses, 800-2000 IU per day, are safe in pregnancy.

Cholecalciferol was first described in 1936. It is on the World Health Organization's List of Essential Medicines. In 2020, it was the 60th most commonly prescribed medication in the United States, with more than 11 million prescriptions. Cholecalciferol is available as a generic medication and over the counter.

Cholecalciferol is a form of vitamin D which is naturally synthesized in skin and functions as a pro-hormone, being converted to calcitriol. This is important for maintaining calcium levels and promoting bone health and development. As a medication, cholecalciferol may be taken as a dietary supplement to prevent or to treat vitamin D deficiency. One gram is 40,000,000 (40×106) IU, equivalently 1 IU is 0.025 μg or 25 ng. Dietary reference intake values for vitamin D (cholecalciferol and/or ergocalciferol) have been established and recommendations vary depending on the country.

In the US: 15 μg/d (600 IU per day) for all individuals (males, females, pregnant/lactating women) between the ages of 1 and 70 years old, inclusive. For all individuals older than 70 years, 20 μg/d (800 IU per day) is recommended. In the EU (except France): 20 μg/d (800 IU per day) and in France: 25 μg/d (1000 IU per day).

Low levels of vitamin D are more commonly found in individuals living in northern latitudes, or with other reasons for a lack of regular sun exposure, including being housebound, frail, elderly, obese, having darker skin, or wearing clothes that cover most of the skin. Supplements are recommended for these groups of people.

The Institute of Medicine in 2010 recommended a maximum uptake of vitamin D of 4,000 IU/day, finding that the dose for lowest observed adverse effect level is 40,000 IU daily for at least 12 weeks, and that there was a single case of toxicity above 10,000 IU after more than 7 years of daily intake; this case of toxicity occurred in circumstances that have led other researchers to dispute it as a credible case to consider when making vitamin D intake recommendations. Patients with severe vitamin D deficiency will require treatment with a loading dose; its magnitude can be calculated based on the actual serum 25-hydroxy-vitamin D level and body weight.

There are conflicting reports concerning the relative effectiveness of cholecalciferol (D₃) versus ergocalciferol (D₂), with some studies suggesting less efficacy of D₂, and others showing no difference. There are differences in absorption, binding and inactivation of the two forms, with evidence usually favoring cholecalciferol in raising levels in blood, although more research is needed.

A much less common use of cholecalciferol therapy in rickets utilizes a single large dose and has been called stoss therapy. Treatment is given either orally or by intramuscular injection of 300,000 IU (7,500 μg) to 500,000 IU (12,500 μg=12.5 mg), in a single dose, or sometimes in two to four divided doses. There are concerns about the safety of such large doses.

Vitamin D₃ is produced through the action of ultraviolet radiation (UV) on the provitamin 7-dehydrocholesterol and is the major form of vitamin D in the human body. Vitamin D₃ is made in human skin and provides about 90% of vitamin D for humans. Transformation of 7-dehydrocholesterol to vitamin D₃ occurs in two steps. First, 7-dehydrocholesterol is photolyzed by ultraviolet light in a synchronous ring-opening electrocycling reaction with six electrons. The product is previtamin D₃. Second, previtamin D₃ spontaneously isomerizes to vitamin D₃ (cholecalciferol). Transformation of previtamin D₃ to vitamin D₃ in organic solvent takes approximately 12 days to complete at room temperature. Conversion of previtamin D₃ to vitamin D₃ in the skin is about 10 times faster than in organic solvents.

The conversion of ergosterol and 7-dehydrocholesterol to vitamin D₃ involves the opening of the sterol B-ring by ultraviolet (UV) activation of the conjugated dienes. Absorption of UV energy activates the molecule, and its π→π* excitation (250-310 nm absorption, λmax=291 nm, ε=12,000) opens the 9,10 bonds and produces previtamin D₂ or previtamin D₂. Resulting in the formation of (Z)-hexadiene, which is vitamin D₃. UV irradiation of 7-dehydrocholesterol or ergosterol results in a steady decrease in the concentration of provitamins, yielding primarily previtamin D initially. When the provitamin level drops below about 10%, the previtamin level reaches a maximum. The concentration of previtamin decreases as it is converted to vitamin D, tachysterol and lumisterol, but subsequent irradiation increases the concentration of previtamin. Temperature, light frequency, irradiation time and substrate concentration all affect the product ratio. The conversion of previtamin D by thermal isomerization at temperatures below 80° C. to give cis-vitamin (ergocalciferol) or cholecalciferol involves an equilibrium as shown in FIG. 3.

Industrially, vitamin D₃ is usually synthesized through photochemical reactions. The synthesis process generally starts from cholesterol, which is first converted into 7-dehydrocholesterol through a series of chemical reactions, and then undergoes a ring-opening reaction to generate pre-vitamin D₃ through sunlight or ultraviolet irradiation, and then undergoes heat and isomerization, and finally converts it into vitamin D₃.

The present invention provides synthetic methods and preparation of cholecalciferol (vitamin D₃) from cholesterol, which is a by-product of the Omega-3 oil production process. According to the invention, dehydro-cholesterol must be prepared and subjected to UV radiation (253 nm). Accordingly, two new synthetic alternatives are provided; where in the first consists of processes of oxidation-reduction of cholesterol to form dehydro-cholesterol and subsequent UV radiation; which route involves six steps (see scheme 1). The second transformation involves the generation of vitamin D₃ by means of a bromination method and a photocatalytic reaction using UV radiation; which alternative requires four steps (see scheme 2).

It should be noted that during each synthetic stage, the solvents used are recycled or recovered; this means that the processes described in the present invention are economical and environmentally friendly. On the other hand, thanks to the experimental data obtained, it was observed that the oxidation-reduction synthesis involves more reaction time and similar yields than the bromination-based approach. However, both synthetic options provide good yields, less synthetic drawbacks, economy and less damage to the environment compared to the currently reported methods. Therefore, the techniques described in this invention become industrially scalable procedures for the production of vitamin D₃.

### BRIEF DESCRIPTION OF THE FIGURES

FIG. 1 is a graphical description of extracting cholesterol from fish oil and using it as a starting material for the synthesis of Vitamin D₃.

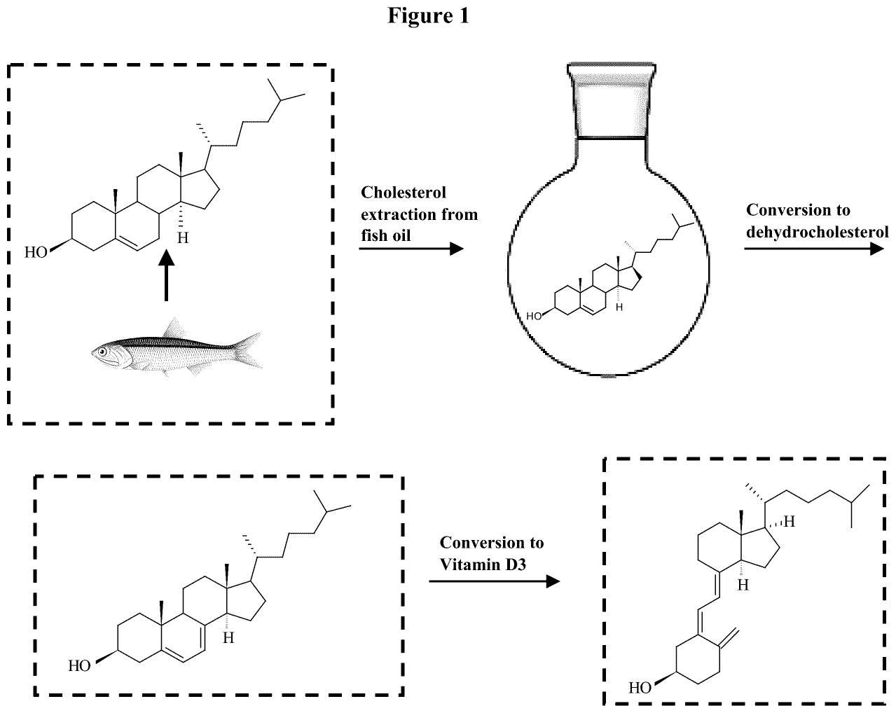

FIG. 2 is the process diagram for step (a) which is the synthesis of 3-acetate-cholesterol.

FIG. 3 depicts a general scheme for the photoconversion and thermo-isomerization of Vitamin D from its precursor.

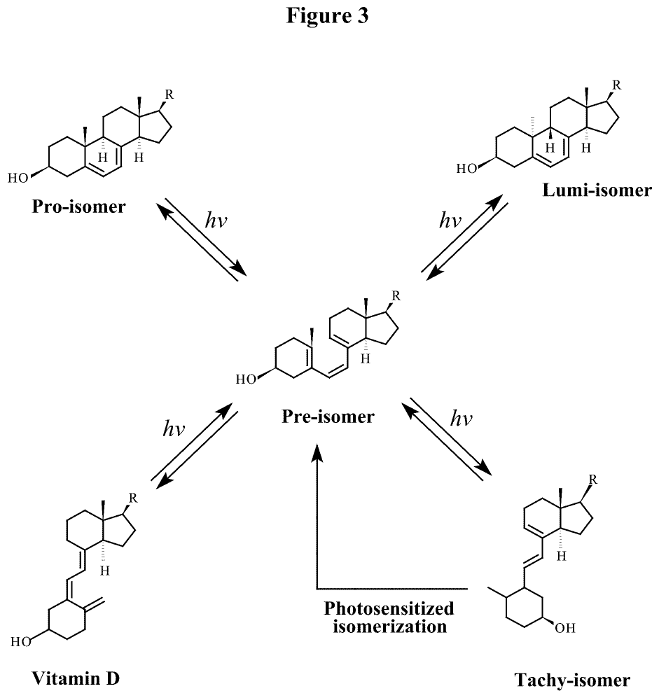

### SUMMARY OF THE INVENTION

The invention provides processes for making Vitamin D₃ starting from cholesterol derived from fish oil. The first process comprises the following steps:
(a) reacting cholesterol with acetic anhydride in a solvent using dimethylamino pyridine as a catalyst to produce the acetylated cholesterol;
(b) reacting the acetylated cholesterol of step (a) with chromium trioxide to produce 3-acetate-7-oxocholesterol;
(c) reacting the 3-acetate-7-oxo-cholesterol of step (b) with p-toluenesulfonyl hydrazide to produce 3-acetate-7-tosylhydrazonecholesterol;
(d) reacting the 3-acetate-7-tosylhydrazonecholesterol of step (c) with lithium hydride to produce 3-acetate-dehydrocholesterol;
(e) reacting the 3-acetate-dehydrocholesterol of step (d) with lithium aluminum tetrahydride to produce 7-dehydrocholesterol; and
(f) irradiating the 7-dehydrocholesterol of step (e) at a wavelength of 254 nm to produce the vitamin D₃.

The second process also starts with cholesterol derived from fish oil. The second process comprises the following steps:
(a) reacting cholesterol with acetic anhydride in a solvent using dimethylamino pyridine as a catalyst to produce the acetylated cholesterol;
(b) reacting the acetylated cholesterol with dibromantin or N-bromosuccinimide in the presence of azobisisobutyronitrile followed by reaction with tertabutylammonium bromide and then tetrabutylammonium fluoride to produce 3-acetate dehydrocholesterol;
(c) reacting the 3-acetate-dehydrocholesterol of step (b) with lithium aluminum tetrahydride to produce 7-dehydrocholesterol; and
(d) irradiating the 7-dehydrocholesterol of step (c) at a wavelength of 254 nm to produce the vitamin D₃.

### DETAILED DESCRIPTION OF THE INVENTION

The present invention will now be described more fully hereinafter with reference to the accompanying drawings and schemes, in which preferred embodiments of the invention are shown. Unless otherwise defined, all technical and scientific terms used herein have the same meaning as commonly understood by one of ordinary skill in the art to which this invention pertains.

The invention provides two processes for making Vitamin D₃ starting from cholesterol derived from fish oil. The cholesterol from the fish oil is manufactured by a process comprising the steps of transesterifying refined oil using a basic catalyst; obtaining a stream concentrated in cholesterol and another stream concentrated in omega-3 fatty acids EPA and DHA by means of molecular distillation; and saponifying the cholesterol-rich stream using bases such as potassium hydroxide, calcium hydroxide, sodium hydroxide or calcium chloride. The mixture is subsequently saponified over a range of time and temperature, with agitation and constant reflux. Multiple extractions are carried out using solvents such as toluene, ethylene dichloride, methylene chloride, diethyl ether, petroleum ether, acetone, benzene or a hexane, and centrifugations are performed to obtain the unsaponifiable matter. This last stream is crystallised using acetone, benzene, toluene, diethyl ether, petroleum ether or a hexane to obtain cholesterol with at least 95% purity. In addition, the soap obtained from saponification is transformed into a form of ethyl ester by applying breakdown reactions and acidic esterification. Full details of the cholesterol production can be found in copending application U.S. Ser. No. 18/029,269 filed Mar. 29, 2023 (corresponding to PCT/IB2020/059184); the contents of which are incorporated by reference in their entirety.

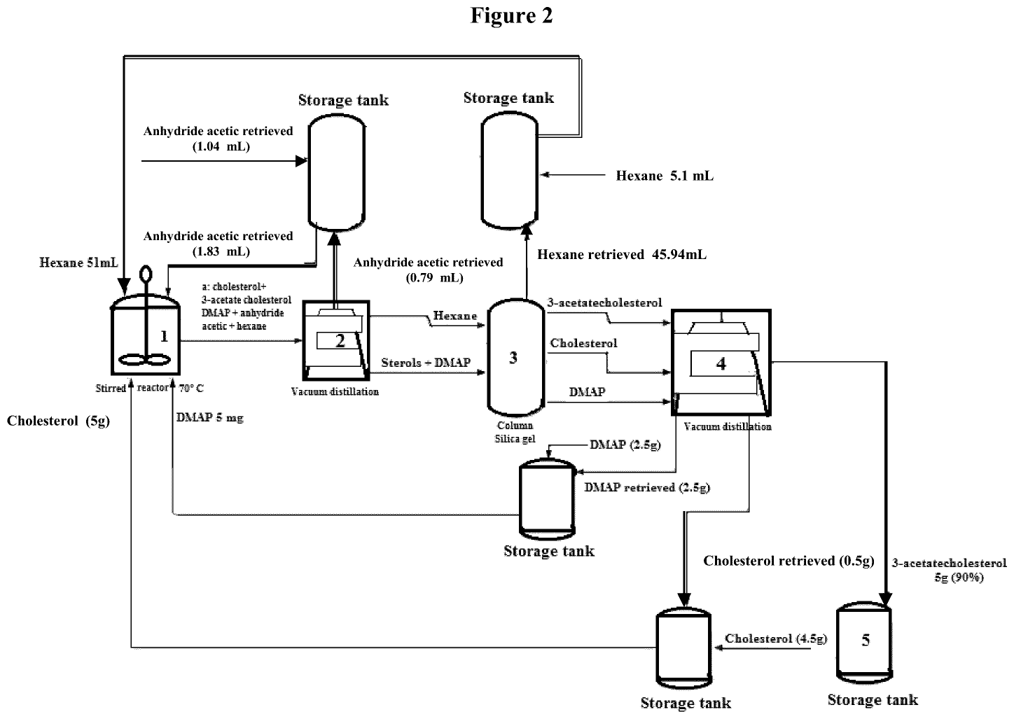

The first process comprises steps (a) through (f). The process is shown structurally in Scheme 1. The synthetic process involves oxidation-reduction, starting with the preparation of cholesterol 3-acetate (compound 1, step a). To accomplish step (a), a solution of cholesterol is prepared in hexane solvent, then acetic anhydride is added in about a 20-30% excess on an equimolar basis and then small amounts of DMAP (4-(Dimethylamino)-pyridine, is added as a nucleophilic acylation catalyst. The reaction mixture is subjected to reflux heating for 4-5 h until the starting reagents are not detected by TLC (thin layer chromatography, using an 8:1 v/v mixture of n-hexane/ethyl acetate as eluent). The reaction crude is allowed to cool to room temperature, followed a distillation process is carried out to recover the remaining acetic acid and the hexane used as solvent, which are recirculated to the process to be used in the reaction step (see FIG. 2). The acquired white solid is purified by column chromatography using silica gel as stationary phase and mixture of hexane and ethyl acetate (8:1) as eluent to obtain compound 2 in 90% yield (Zhao et al., 2014). Unreacted cholesterol (10% of the sample) and 50% DMAP are recovered and used again in the reaction.

Other solvents that can be used in step (a) are selected from the group consisting of benzene, ethyl acetate, chloroform, methylene chloride, acetone, diethyl ether, diisopropyl ether and cyclohexane. Also as an alternative triethyl amine can be used as a catalyst alone or also in combination with DMAP.

The second transformation (step b) consists of the oxidation of cholesteryl 3-acetate to obtain 3-acetate-7-oxocholesterol (compound 3). For this process, all reagents must be dried as shown in Table 1.

TABLE 1
Drying process of the reagents for
the preparation of Collins' reagent.

| Reagent | Drying method |
|---|---|
| CrO₃ | 15 g of the compound are weighed into a glass container |
| (chromium | and placed in an oven at 100° C. under vacuum (−25 mm |
| oxide VI) | Hg) for 24 hours. |
| Pyridine | 33 mL of pyridine is measured and deposited in an amber |
| | flask, then 500 mg of previously activated 4 Å molecular |
| | sieve (heated in oven at 110° C. for 4 h) is added, the |
| | sieve must not be removed from the liquid at any time. |
| Dichloro- | 350 mL of the solvent is measured and deposited in an |
| methane | amber flask, then 800 mg of previously activated 4 Å |
| | molecular sieve (heated in oven at 110° C. for 4 h) is |
| | added, the sieve must not separate from the liquid at any |
| | time. |

NOTE:
It is advisable to use a desiccant filter (either molecular sieve, zeolite or activated alumina), as it is much more economical and environmentally friendly.

With the reagents dry, one proceeds to the preparation of the Collins reagent according to the reported literature (Fullerton & Chen, 1976), which consists of preparing a solution of pyridine in dichloromethane as a solvent and cooling it in an ice bath with salt (NaCl) and alcohol (ethanol). The solution is stirred for 30 min under nitrogen atmosphere and, consecutively, the chromium oxide (VI, CrO3) is added. The solution is stirred for 25 min until it acquires a deep orange coloration, then the ice bath is removed and the solution is stirred for another 10 min. Other solvents for Collins reagents may be used such as chlorofrom, carbon tertrachloride and dichloroethane.

With the above Collins reagent prepared, cholesterol acetate is dissolved in dichloromethane and the solution is added to the previously prepared reagent. Upon addition of the acetate-cholesterol, a tarry precipitate should immediately begin to form on the sides and bottom of the flask. After stirring for about 20 h at room temperature, the solution is filtered under vacuum and the tarry deposit in the flask is washed with dichloromethane, again filtered under vacuum. The filtrate liquid is distilled under vacuum, in order to recover the remaining dichloromethane and pyridine, these solvents are passed through a silica gel column and used again in the process. The residual oil is purified by column chromatography using silica gel as stationary phase and as eluent a mixture of hexane and ethyl acetate (5/1 ratio). Finally, about 10% of the starting material (3-acetate-cholesterol), a by-product (white solid 15%) and the target compound were recovered in 68-75% yield. The obtained compound was fully identified by melting point, infrared spectroscopy (IR) and 1H NMR proton nuclear magnetic resonance (Fullerton & Chen, 1976).

Continuing with the synthetic process to step (c), to obtain the compound 3-acetate-7-tosylhydrazoncholesterol (compound 4), p-toluenesulfonyl hydrazide and compound 3 are added to methanol as solvent, and likewise, glacial acetic acid is then added. The mixture is heated at reflux for 7-8 h until no TLC of the starting materials are detected (using ethyl acetate 5/1 hexane as mobile phase). Once the time is completed, the reaction is allowed to cool down to room temperature and poured over cold water, the mixture is stirred for 15 min, the yellow solid formed is recovered by vacuum filtration and the filtered liquid is distilled off under reduced pressure; this in order to recover methanol and acetic acid (the recovered solvents must be filtered over silica gel). As for the acquired solid, it is dried at room temperature. Alternative solvents include ethanol and propanol.

It should be noted that the synthesized compound does not require any purification for the further structural modification. However, a purification by column chromatography was performed using silica gel as a stationary phase and as eluent a hexane/ethyl acetate mixture (2/1 ratio), in order to verify the yield of the pure product. Thanks to the above process, a white solid (compound 4) was obtained with a yield of 90%, better than that reported in the literature (Yablonskaya & Segal, 1973). The obtained compound was identified by melting point 147-149° C.(Yablonskaya & Segal, 1973) as reported in the literature.

As for the preparation of 3-acetate-dehydrocholesterol (compound 5) as shown in step (d), the Bamford-Stevens reaction is implemented using a salt hydride. On the basis of the above, to a solution of compound 4 in toluene as a solvent, LiH is rapidly added, and the mixture is subjected to reflux for 5 h, after which the reaction mixture is cooled and filtered under vacuum. The filtered liquid is cooled in an ice bath and neutralized (pH=7) with a 2% solution of sulfuric acid (H₂SO₄), filtered and then distilled under vacuum to recover the toluene (the recovered solvent is passed through a silica gel filter). The reaction crude obtained does not require any purification for the following synthetic process. However, a column chromatographic purification was performed using silica gel as stationary phase and as eluent a hexane-ethyl acetate mixture (ratio 25/1), to verify the yield of the pure product. Finally, a white solid (compound 5) with a yield between 35-40% is obtained. The obtained compound was identified with a melting point of 128-130° C.(Yablonskaya & Segal, 1973).

Regarding the preparation of 7-dehydrocholesterol (step e, compound 6), a reduction of product 5 is made by a modification of the method reported by Zhao et al. (Zhao et al., 2014). Given the above, a solution of derivative 5 is prepared in THF (tetrahydrofuran), then the solution is cooled in an ice bath. Carefully, there is added LiAlH₄ (lithium aluminum tetrahydride) and leave it in stirring for 1 h. After this time, leave it again in stirring at room temperature for 2 h more. After completion of the reaction, verified by TLC (using a 2:1 v/v n-hexane/ethyl acetate mixture as eluent), the mixture is neutralized (pH=7) with a 5% solution of HCl, filtered and the mixture is subjected to vacuum distillation (the recovered THF must be filtered over silica gel before being reused in the process). The recovered solid is washed with water, dried at room temperature and purified by recrystallization in ethanol. Finally, a white solid (compound 6) is obtained in 75-80% yield. The obtained compound is identified by melting point 150° C., according to Sigma-Aldrich 148-152ºC. The 7-Dehydrocholesterol can also be purified from a solvent selected from the group consisting of methanol; ethanol or 2-propanol, Acetone, 2-Butanone, Methyl isobutylketone or Di-isobutylketone or from a mixture of solvents like Dichloromethane and Methanol or Toluene and methanol or Methanol and Diisopropyl ether.

To finalize the synthetic process (step f) a photocatalytic process is conducted based on the processes reported in the scientific literature (Lin et al, 2018; Niu et al, 2021; Zmijewski et al, 2008). To carry out this reaction a solution of the previously synthesized 7-dehydrocholesterol is prepared in ethyl ether as a solvent, the mixture is deposited in a quartz tube and irradiated at a wavelength of 254 nm (724 Lux) at a temperature of 65° C., for 15 min. The reaction is monitored by TLC (5:1 hexane/acetate mobile phase) until the starting reagents are no longer present. At the end of the process, the formation of 4 compounds (identified by TLC) with similar RF (retention point) is observed. The mixture is removed from the photocatalytic reactor and subjected to vacuum evaporation, passed through a silica gel filter (to be reused) and the solid obtained is analyzed by HPLC chromatography using vitamin D₃ as a standard, thanks to this technique it was possible to confirm that the protocol described here provides cholecalciferol (vitamin D₃, compound 7) with an abundance of 40%. Surprisingly, the formation of three structural isomers (by analyzing its chemical structure) is evidenced.

The irradiation step can also be conducted in a solvent selected from the group consisting of an alcohol, an alkene, a polar solvent, a nonpolar solvent, a cycloalkane, an ether, a carboxylic acid ester, and an aromatic solvent. Among the above family of solvents, one can use acetonitrile, toluene, pyridine, trichloroethylene, acetone, 1,2-ethanediol, ethanol, methanol, isopropanol, diethyl ether, ethyl acetate, dimethylsulfoxide, dimethyl-formamide, diethylamine, triethylamine, chloroform, anisole, benzene, 1-butanol, chloroform, cyclohexane, acetic acid butyl ester, hexane, 2-propanol, 1-hexene, naphthalene, tetrahydrofuran, m-xylene, p-xylene, o-xylene, n-methyl-2-pyrrolidone, 1,3-butadiene, and hexadecane, or mixtures thereof.

The irradiation of the starting material in a solution can also be done with light in the wavelength range 245-360 nanometers (nm) to obtain a product containing vitamin-D₃.

The second process of the invention is based on an adaptation to the method reported by Lin and co-workers (Lin et al, 2018). The synthetic process starts with the preparation of 3-acetocholesterol (step a, as described above in the first process) using cholesterol obtained from Naturmega as the raw material.

The second process as shown in Scheme 2 includes less steps. Step b as shown in Scheme 2 consists of preparing a solution of cholesteryl acetate in benzene-hexane 1:1 as solvent. To this solution there is added dibromantin, azobisisobutyronitrile and sodium bicarbonate. The mixture is heated under reflux under nitrogen for 10 min in a preheated vessel at 100° C. in an oil bath. The reaction crude is then cooled in an ice bath. Possible insoluble material is removed by suction filtration and then the filtered liquid is distilled under vacuum (to recover the solvents benzene and hexane). The reaction crude obtained is dissolved in anhydrous tetrahydrofuran and then tetrabutylammonium bromide is added. The resulting solution is stirred for 75 min under nitrogen at room temperature. To this reaction mixture there is added tetrabutylammonium fluoride. The mixture is stirred for 50 min, continuously rotary-evaporated at 40-45° C. (to recover THF) until a brown solid is obtained. A solution of this solid is washed in ethyl acetate with water and dried over anhydrous Na₂SO₄. According to the literature, the evaporation of the solvent gives 2.03 g, and this crude product must be purified by column chromatography using as eluent hexane-ethyl acetate 95:5; to give a yield of 40-50%. The reactions with the tertabutylammonium bromide and tetrabutylammonium fluoride can also be conducted in other solvents selected from the group consisting of toluene, dimethyl formamide, acetone, a mixture of two of these solvents and a mixture of one of these solvents with an alkane having from 6 to 8 carbon atoms. Mixtures of tetrahydrofuran and hexane or heptane as a solvent can also be used. Additionally, the dehydrobromination reaction is carried out in tetrahydrofuran or in a mixture of tetrahydrofuran and hexane or heptane as a solvent at a reaction temperature between 20° C. and 70° C., preferably at approximately 25° C. Similarly, the tetrabutyl moieties can be replaced with other tertaalkyl groups having 1-3 and 5-15 carbon atoms.

Once the 3-acetate-dehydrocholesterol (compound 5) is prepared, the deprotection of product 5 using LiAlH₄ follows (for this situation the same procedure stipulated in step e of scheme 1 is performed), and finally, the photocatalytic process as described in scheme 1 is replicated (step f). The synthetic scheme described above, involves fewer reaction steps, less risk and shorter reaction times.

### Description of the Synthetic Process

Scheme 1 below is a synthetic route for obtaining vitamin D₃, by oxidation-reduction.

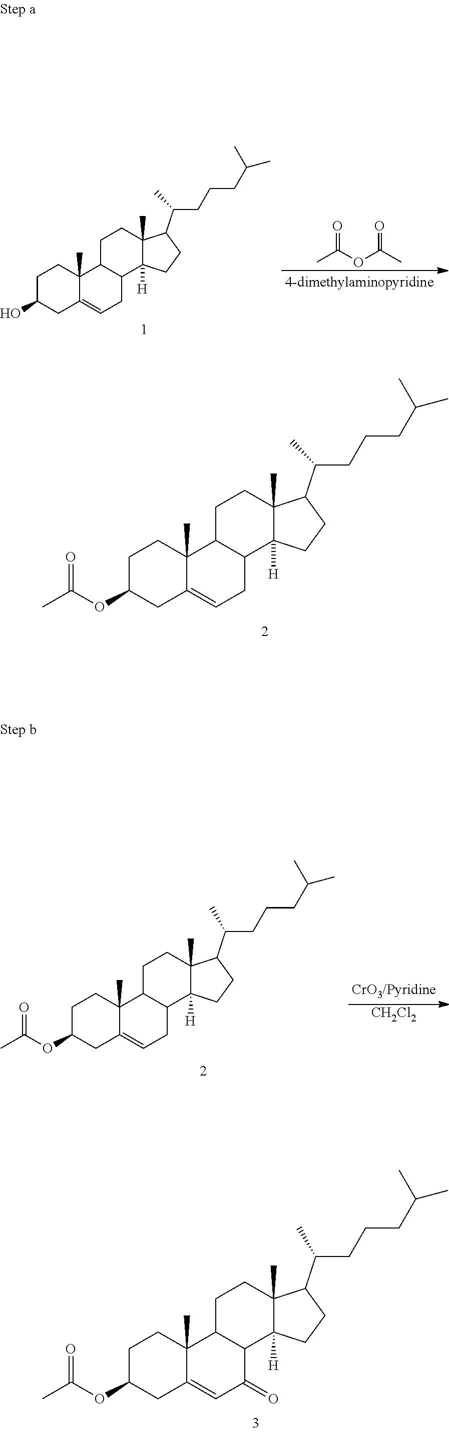

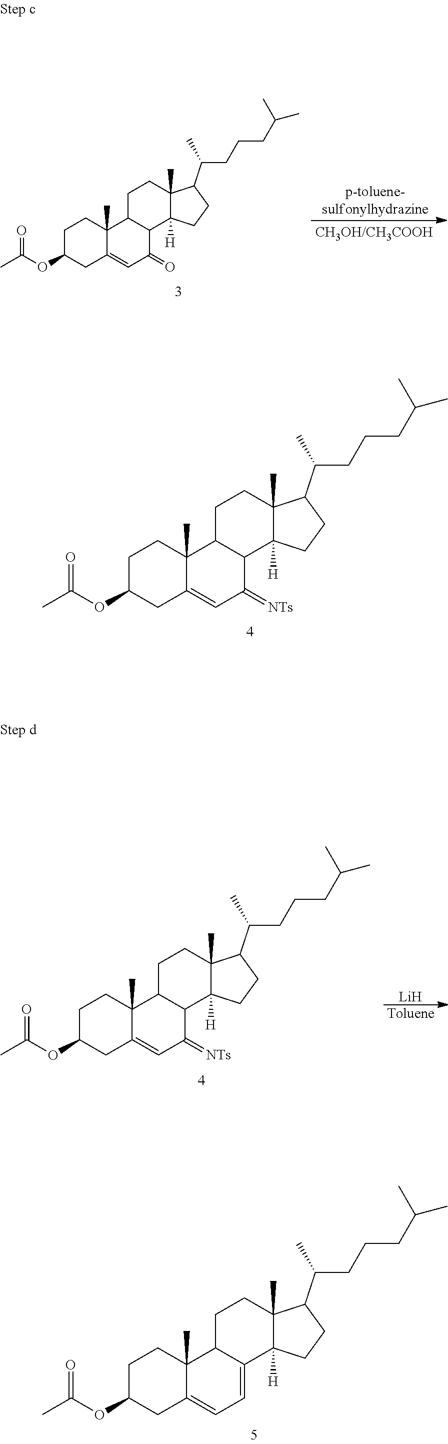

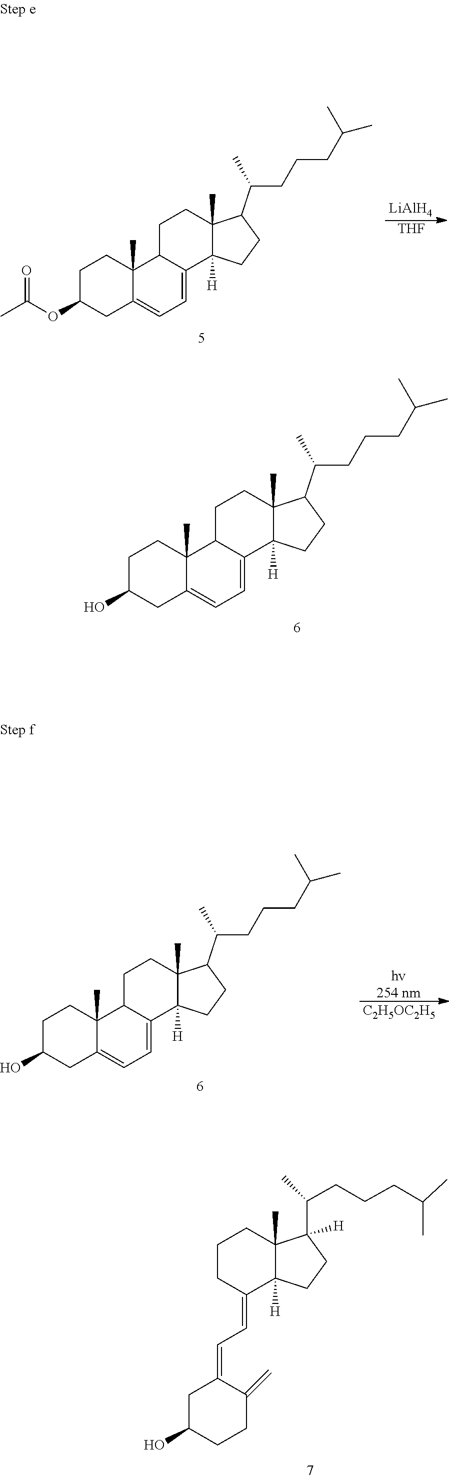

Scheme 1 shows the synthetic process by oxidation-reduction, starting with the preparation of cholesterol 3-acetate (compound 1, step a).

The following examples are intended to demonstrate the usefulness of preferred embodiments of the present invention and should not be considered to limit its scope or applicability in any way.

### Example I

A solution of cholesterol (5 g, 12.93 mmol) is prepared in 51 mL hexane, then acetic anhydride (1.83 mL, 15.48 mmol) and DMAP (4-(Dimethylamino)-pyridine, 5 mg, 0.040 mmol)) is added. The reaction mixture is subjected to reflux heating for 4-5 h until the starting reagents are not detected by TLC (thin layer chromatography, using an 8:1 v/v mixture of n-hexane/ethyl acetate as eluent). The reaction crude is allowed to cool to room temperature, followed a distillation process is carried out to recover the remaining acetic acid and the hexane used as solvent, which are recirculated to the process to be used in the reaction step (see FIG. 2). The acquired white solid is purified by column chromatography using silica gel as stationary phase and mixture of hexane and ethyl acetate (8:1) as eluent to obtain compound 2 in 90% yield (Zhao et al., 2014). Unreacted cholesterol (10% of the sample) and 50% DMAP are recovered and used again in the reaction.

### Example II

The second transformation (step b) consists of the oxidation of cholesteryl 3-acetate to obtain 3-acetate-7-oxocholesterol (compound 3). For this process, all reagents must be dried, see Table 1.

TABLE 1
Drying process of the reagents for
the preparation of Collins' reagent.

| Reagent | Drying method |
|---|---|
| CrO₃ | 15 g of the compound are weighed into a glass container |
| (chromium | and placed in an oven at 100° C. under vacuum (−25 mm |
| oxide VI) | Hg) for 24 hours. |
| Pyridine | 33 mL of pyridine is measured and deposited in an amber |
| | flask, then 500 mg of previously activated 4 Å molecular |
| | sieve (heated in oven at 110° C. for 4 h) is added, the |
| | sieve must not be removed from the liquid at any time. |
| Dichloro- | 350 mL of the solvent is measured and deposited in an |
| methane | amber flask, then 800 mg of previously activated 4 Å |
| | molecular sieve (heated in oven at 110° C. for 4 h) is |
| | added, the sieve must not separate from the liquid at any |
| | time. |

NOTE:
It is advisable to use a desiccant filter (either molecular sieve, zeolite or activated alumina), as it is much more economical and environmentally friendly.

With the reagents dry, we proceed to the preparation of the Collins reagent according to the reported literature (Fullerton & Chen, 1976), this consists of preparing a solution of pyridine (25 mL, 310 mmol) in 150 mL of dichloromethane and cooling it in an ice bath with salt (NaCl) and alcohol (ethanol). The solution is stirred for 30 min under nitrogen atmosphere and, consecutively, 15 g (150 mmol) chromium oxide (VI, Cr03) is added. The solution is stirred for 25 min until it acquires a deep orange coloration, then the ice bath is removed and the solution is stirred for another 10 min.

With the Collins reagent prepared, 5 g (11.66 mmol) of cholesterol acetate is dissolved in 5 mL of dichloromethane, the solution is added to the previously prepared reagent. Upon addition of the acetate-cholesterol, a tarry precipitate should immediately begin to form on the sides and bottom of the flask. After stirring for 20 h at room temperature, the solution is filtered under vacuum and the tarry deposit in the flask is washed with dichloromethane, again filtered under vacuum. The filtrate liquid is distilled under vacuum, in order to recover the remaining dichloromethane and pyridine, these solvents are passed through a silica gel column and used again in the process. The residual oil is purified by column chromatography using silica gel as stationary phase and as eluent a mixture of hexane and ethyl acetate (5/1 ratio). Finally, 10% of the starting material (3-acetate-cholesterol), a by-product (white solid 15%) and the target compound were recovered in 68-75% yield. The obtained compound was identified by melting point, infrared spectroscopy (IR) and 1H NMR proton nuclear magnetic resonance (Fullerton & Chen, 1976).

### Example III

Continuing with the synthetic process, to obtain the compound 3-acetate-7-tosylhydrazoncholesterol (compound 4), 1.2 g (2.5 mmol) of p-toluensulfonylhydrazine, 1.2 g of compound 3 (1 mmol) are added to 30 mL of methanol, likewise, 0.8 mL of glacial acetic acid is added. The mixture is heated at reflux for 7-8 h until no TLC of the starting materials are detected (using ethyl acetate 5/1 hexane as mobile phase). Once the time is completed, the reaction is allowed to cool down to room temperature and poured over cold water, the mixture is stirred for 15 min, the yellow solid formed is recovered by vacuum filtration and the filtered liquid is distilled off under reduced pressure; this in order to recover methanol and acetic acid (the recovered solvents must be filtered over silica gel). As for the acquired solid, it is dried at room temperature.

It should be noted that the synthesized compound does not require any purification for the further structural modification. However, a purification by column chromatography was performed using silica gel as stationary phase and as eluent a hexane/ethyl acetate mixture (2/1 ratio), in order to verify the yield of the pure product. Thanks to the above process, a white solid (compound 4) was obtained with a yield of 90%, better than that reported in the literature (Yablonskaya & Segal, 1973). The obtained compound was identified by melting point 147-149° C.(Yablonskaya & Segal, 1973) as reported in the literature.

### Example IV

As for the preparation of 3-acetate-dehydrocholesterol (compound 5), the Bamford-Stevens reaction is implemented using a salt hydride (step d), on the basis of the above, a solution of compound 4 (1 g, 1.62 mmol) is prepared in 20 mL of toluene, LiH 0.971 g is rapidly added, the mixture is subjected to reflux for 5 h, after which the reaction mixture is cooled and filtered under vacuum. The filtered liquid is cooled in an ice bath and neutralized (pH=7) with a 2% solution of sulfuric acid (H₂SO₄), filtered and then distilled under vacuum to recover the toluene (the recovered solvent is passed through a silica gel filter). The reaction crude obtained does not require any purification for the following synthetic process. However, a column chromatographic purification was performed using silica gel as stationary phase and as eluent a hexane-ethyl acetate mixture (ratio 25/1), to verify the yield of the pure product. Finally, a white solid (compound 5) with a yield between 35-40% is obtained. The obtained compound was identified with a melting point of 128-130° C.(Yablonskaya & Segal, 1973).

### Example V

Regarding the preparation of 7-dehydrocholesterol (step e, compound 6), a reduction of product 5 is carried out by a modification of the method reported by Zhao et al. (Zhao et al., 2014). Given the above, a solution of derivative 5 (300 mg) is prepared in THF (10 mL, tetrahydrofuran), the solution is cooled in an ice bath. Carefully add LiAlH₄ (lithium aluminum tetrahydride) and leave it in stirring for 1 h. After this time, leave it again in stirring at room temperature for 2 h more. After completion of the reaction, verified by TLC (using a 2:1 v/v n-hexane/ethyl acetate mixture as eluent), the mixture is neutralized (pH=7) with a 5% solution of HCl (15 mL), filtered and the mixture is subjected to vacuum distillation (the recovered THF must be filtered over silica gel before being reused in the process). The recovered solid is washed with water, dried at room temperature and purified by recrystallization in ethanol. Finally, a white solid (compound 6) is obtained in 75-80% yield. The obtained compound is identified by melting point 150° C., according to Sigma-Aldrich 148-152ºC.

### Example VI

To finalize the synthetic process (step f) a photocatalytic process is executed based on the processes reported in the scientific literature (Lin et al, 2018; Niu et al, 2021; Zmijewski et al, 2008), to carry out this reaction a solution of 50 mg of the previously synthesized 7-dehydrocholesterol is prepared in 10 mL of ethyl ether, the mixture is deposited in a quartz tube and irradiated at a wavelength of 254 nm (724 Lux) at a temperature of 65° C., for 15 min. The reaction is monitored by TLC (5:1 hexane/acetate mobile phase) until the starting reagents are no longer present; at the end of the process, the formation of 4 compounds (identified by TLC) with similar RF (retention point) is observed. The mixture is removed from the photocatalytic reactor and subjected to vacuum evaporation, passed through a silica gel filter (to be reused) and the solid obtained is analyzed by HPLC chromatography using vitamin D₃ as a standard, thanks to this technique it was possible to confirm that the protocol described here provides cholecalciferol (vitamin D₃, compound 7) with an abundance of 40%. Surprisingly, the formation of three structural isomers (analyzing its chemical structure) is evidenced.

It should be noted that, in each recovery carried out by vacuum distillation, between 85-95% of each solvent is recovered.

To make a brief comparison of the experimental design proposed in this work with other synthetic processes already reported in Table 2, where the differences and advantages of the synthetic route proposed in scheme 1 can be observed.

TABLE 2
Comparison between the proposed synthetic design and
the processes described in each step of the reaction.

| | Advantages of the proposed | |
|---|---|---|
| Article | synthetic process | Remarks |
| Step a (Zhao | Use Naturmega Cholesterol | The authors report a shorter reaction time, |
| et al, 2014) | Lower amount of acetic | however, when monitoring the reaction |
| | anhydride | crude by TLC in the time stipulated in the |
| | Higher yield | document, the presence of the precursor is |
| | Does not involve the use of | appreciated, for this reason it was decided |
| | HCl | to extend the reaction time (85-90% yield). |
| Step b (Fullerton | Use of cholesterol acetate | When performing the process reported by |
| & Chen, 1976) | from the acetylation of | the authors, it is evident that a lower yield |
| | cholesterol Naturmega | of the desired product is obtained. For this |
| | Phosphorus pentoxide and | reason, it was explored to leave the |
| | calcium chloride are not | reaction for 20, 24, 36 and 48 hours, where |
| | necessary for drying. | it was evidenced that the best reaction time |
| | The n-hexane/acetate mixture | is at 20 hours. In addition, treatment with a |
| | is less volatile and less toxic. | hexane/ethyl acetate mixture improves the |
| | | yield. It should also be taken into account |
| | | that the humidity of the city of Barranquilla |
| | | is high, which has a negative influence on |
| | | the reaction since it is quite susceptible to |
| | | water (yields between 60-65%). |
| Step b | Use of cholesterol acetate | The process mentioned by the authors is |
| (Chidambaram & | from the acetylation of | not so recommendable since the use of the |
| Chandrasekaran, | cholesterol Naturmega | different reagents is quite dangerous; |
| 1987) | No benzene required | moreover, they are expensive and difficult |
| | Does not require dry pyridine | to obtain. |
| | chromate (solid) | |
| | No tert-butyl-hydroperoxide | |
| | required | |
| | No zeolite required | |
| Step b | Use of cholesterol acetate | The reaction described by the authors is |
| (Dauben et al., | from Naturmega cholesterol | very similar to the one presented in this |
| 1969) | acetylation. | article; the difference lies in the in situ way |
| | Same preparation as Collins | in which the reaction is carried out (all at |
| | reagent | the same time). Complementarily, the |
| | No drying of Collins reagent | process described here presents a yield |
| | required | equal or superior to that of the cited article. |
| Step b | Use of cholesterol acetate | The process mentioned by the authors is |
| (Parish & Wei, | from Naturmega cholesterol | not so recommendable since the use of |
| 1987) | acetylation. | aromatic solvents (toluene and benzene) |
| | Does not require heating, | makes the treatment of the reaction |
| | benzene, toluene or HCl. | difficult, besides being highly toxic. |
| | No drying of the Cr-pyridine | |
| | complex required. | |
| Step c | Use of -3-acetate-7- | Although the process described by the |
| (Loncle et al., | oxocholesterol, from | authors was reproduced, good yields (30- |
| 2004) | Naturmega cholesterol. | 40%) were not obtained. Therefore, a |
| | No HCl required | methodology based on a reflux |
| | Much shorter reaction times | condensation reaction using an organic acid |
| | More economical | was designed. So far, no work has been |
| | | found that implements this type of reaction |
| | | for the generation of hydrazones from |
| | | sterols and hydrazines. Surprisingly, it |
| | | generates a yield of 90%, moreover, it does |
| | | not require purification for the following |
| | | structural modification. |
| Step d | Use of 3-acetate-7- | For this synthetic transformation, the |
| (Yablonskaya & | tosylhydrazoncholesterol, by | method described by the authors was |
| Segal, 1973) | Naturmega Cholesterol | followed. Unfortunately, a yield of 35% |
| | | was obtained, which is significantly lower |
| | | than the yield indicated in the article (93% |
| | | yield). We are currently evaluating other |
| | | alternatives to improve the yield of the |
| | | desired product. |
| Step e (Zhao | Use of 3-acetate-7- | For this synthetic modification it was not |
| et al., 2014) | dehydrocholesterol, from | necessary to heat at reflux or stir the |
| | Naturmega cholesterol. | mixture at 0° C. for such a long time, the |
| | No reflux heating required | difference lies in the stirring at room |
| Step e | Use of 3-acetate-7- | temperature for 2 h, the yield obtained |
| (Guo et al., 2003) | dehydrocholesterol, from | ranged between 75-80%. |
| | Naturmega Cholesterol. | |
| | It is not necessary to shake | |
| | for a long time at 0° C. | |
| Step f | Use of 7-dehydrocholesterol, | The photochemical reaction presented in |
| Wenhao Niu- | from Naturmega cholesterol. | this work is quite different from that of the |
| 2021 | Does not require the use of | cited articles, since the target product |
| | methyl tert-butyl ether. | (vitamin D₃) is obtained in a single step. |
| | Does not require the use of | This process consists of subjecting a |
| | inert atmosphere. | solution of product 6 in ethyl ether (see |
| | Does not require low | scheme 1) to Uv radiation (240 nm, 724 |
| | temperatures. | Lux) and heating at 65° C. for 15 min. This |
| | No pressure required. | transformation generates approximately |
| Step f | Use of 3-acetate-7- | 40% of the target product, however, there |
| Michal A. | dehydrocholesterol, from | are indications of the formation of three |
| Zmijewski- 2008 | Naturmega Cholesterol. | more products, which are suspected to be |
| | Does not require the use of | isomers generated by the photoreaction. |
| | dichloromethane. | We are currently analyzing the type of by- |
| | Does not require reflux | products formed. |
| | heating in ethanol for the | |
| | generation of vitamin D₃. | |
| | Requires a reaction time of | |
| | 15 min. | |

In order to explore other synthetic options for obtaining vitamin D₃, another synthetic route is described (see Scheme 2).

Scheme 2 shown below is another synthetic route for obtaining vitamin D₃ by bromination.

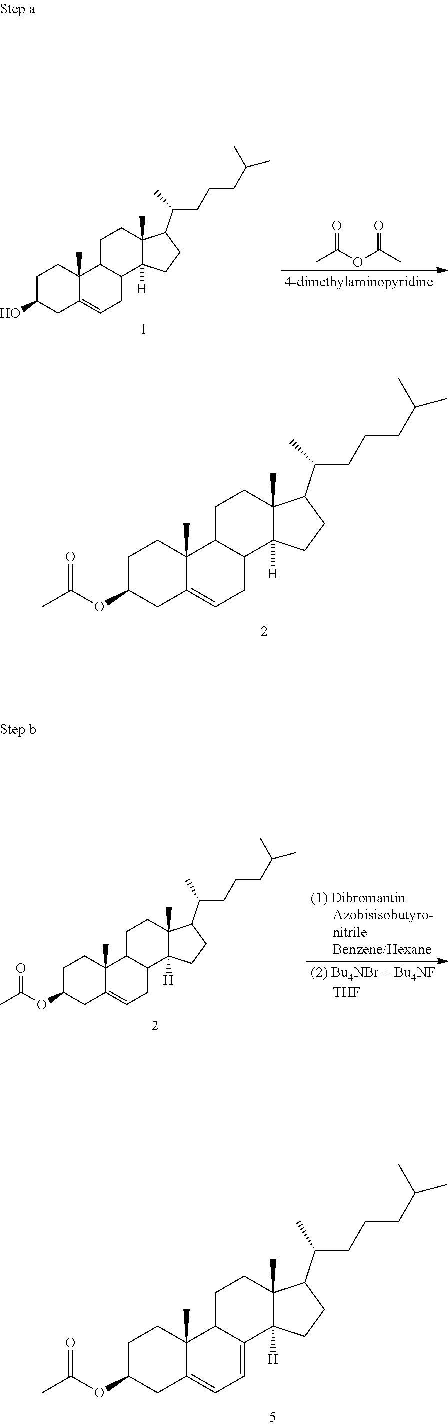

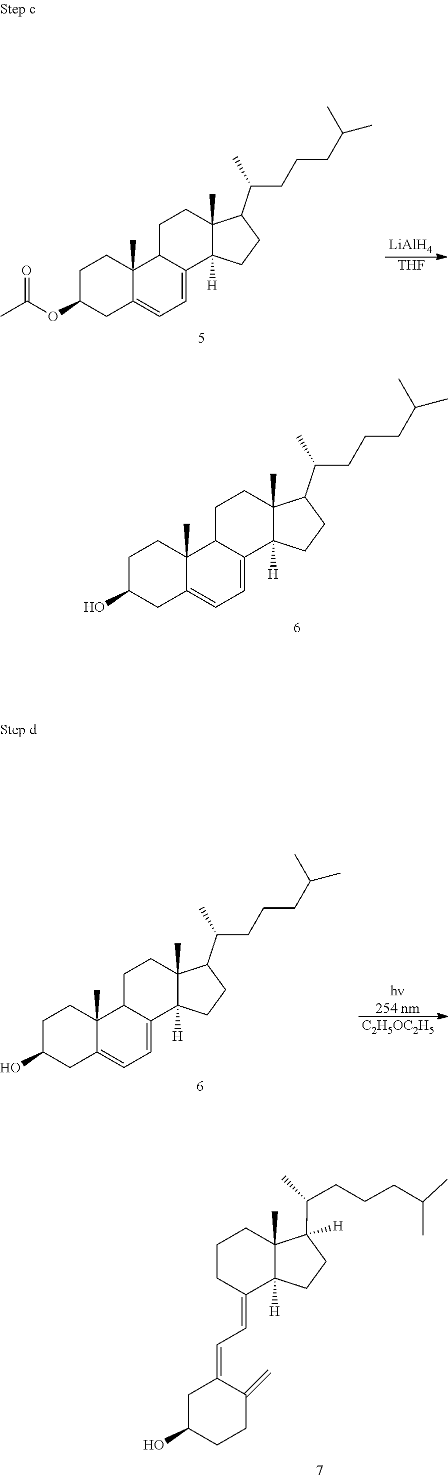

This approach is based on an adaptation to the method reported by Lin and co-workers (Lin et al, 2018). The synthetic process starts with the preparation of 3-acetocholesterol (step a, described above) using cholesterol obtained from Naturmega as raw material.

### Example VII

The second process (step b, Scheme 2) consists of preparing a solution of cholesteryl acetate (2.03 g, 4.8 mmol) in benzene-hexane 1:1 (120 ml), to this solution is added dibromantin (0.84 g, 2.92 mmol) and sodium bicarbonate (2.74 g) and catalytic amounts of AIBN. The mixture is heated under reflux under nitrogen for 10 min in a preheated vessel at 100° C. in an oil bath. The reaction crude is then cooled in an ice bath. Possible insoluble material is removed by suction filtration and then the filtered liquid is distilled under vacuum (to recover the solvents benzene and hexane).

### Example VIII

The reaction crude obtained above in example VII is dissolved in anhydrous tetrahydrofuran (40 ml) and tetrabutylammonium bromide (0.4 g) is added. The resulting solution is stirred for 75 min under nitrogen at room temperature. To this reaction mixture is added tetrabutylammonium fluoride (10 ml, 1 M solution in tetrahydrofuran, 10 mmol). The mixture is stirred for 50 min, continuously rotated-evaporated at 40-45° C.(to recover THF) until a brown solid is obtained. A solution of this solid is washed in ethyl acetate (200 ml) with water (3×50 ml) and dried over anhydrous Na₂SO₄. According to the bibliography the evaporation of the solvent gives 2.03 g, this crude must be purified by column using as eluent hexane-ethyl acetate 95:5. Yield 40-50%.

### Example IX

Once the 3-acetate-dehydrocholesterol (compound 5) is prepared, the deprotection of product 5 using LiAlH₄ follows (for this situation the same procedure stipulated in step e of scheme 1 is performed), finally, the photocatalytic process described in scheme 1 is replicated (step f). The synthetic scheme described above, involves fewer reaction steps, less risk and shorter reaction times.

Based on the modifications made to the processes already reported for obtaining sterols, it was possible to develop two new eco-friendly synthetic alternatives (oxidation-reduction method and bromination method) that allow generating vitamin D₃ with a good yield, using as starting material cholesterol obtained as a by-product of the omega 3 concentrates process carried out by the company Naturmega.

On the other hand, a brief comparison of the proposed methodologies shows that the alternative based on bromination provides the product (vitamin D₃) in less time, represents less toxicity and involves fewer chemical alterations (reaction steps). Moreover, it is important to highlight that in several steps of the synthesis it is possible to reuse some reagents and solvents, making the process environmentally friendly and reducing input costs by recovering them.

All literature and similar materials cited in this application including, but not limited to, patents, patent applications, articles, books, treatises, and internet web pages, regardless of the format of such literature and similar materials, are expressly incorporated by reference in their entirety for any purpose as if they were entirely denoted. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents. In the event that one or more of the incorporated literature and similar materials defines or uses a term in such a way that it contradicts that term's definition in this application, this application controls.

Although the foregoing description contains many specifics, these should not be construed as limiting the scope of the present invention, but merely as providing illustrations of some of the presently preferred embodiments. Similarly, other embodiments may be devised without departing from the spirit or scope of the present invention. Features from different embodiments may be employed in combination. The scope of the invention is, therefore, indicated and limited only by the appended claims and their legal equivalents rather than by the foregoing description. All additions, deletions and modifications to the invention as disclosed herein which fall within the meaning and scope of the claims are to be embraced thereby.

### REFERENCES

1. Chidambaram, N., & Chandrasekaran, S. (1987). Tert-Butyl hydroperoxide-pyridinium dichromate: A convenient reagent system for allylic and benzylic oxidations. The Journal of Organic Chemistry, 52(22), 5048-5051. https://doi.org/10.1021/jo00231a046
2. Dauben, W. G., Lorber, M. E., & Fullerton, D. S. (1969). Allylic oxidation of olefins with chromium trioxide pyridine complex. The Journal of Organic Chemistry, 34(11), 3587-3592. https://doi.org/10.1021/jo01263a079
3. Fullerton, D. S., & Chen, C.-M. (1976). In Situ Allylic Oxidations With Collins Reagent. Synthetic Communications, 6(3), 217-220. https://doi.org/10.1080/00397917608072633
4. Guo, L.-W., Wilson, W. K., Pang, J., & Shackleton, C. H. L. (2003). Chemical synthesis of 7- and 8-dehydro derivatives of pregnane-3,17a,20-triols, potential steroid metabolites in Smith-Lemli-Opitz syndrome. Steroids, 68(1), 31-42. https://doi.org/10.1016/S0039-128X(02)00113-7
5. Hutchins, R. O., Milewski, C. A., & Maryanoff, B. E. (1973). Selective deoxygenation of ketones and aldehydes including hindered systems with sodium cyanoborohydride. Journal of the American Chemical Society, 95(11), 3662-3668. https://doi.org/10.1021/ja00792a033
6. Lin, Z., Marepally, S. R., Goh, E. S. Y., Cheng, C. Y. S., Janjetovic, Z., Kim, T.-K., Miller, D. D., Postlethwaite, A. E., Slominski, A. T., Tuckey, R. C., Peluso-Iltis, C., Rochel, N., & Li, W. (2018). Investigation of 20S-hydroxyvitamin D₃ analogs and their 1a-OH derivatives as potent vitamin D receptor agonists with anti-inflammatory activities. Scientific Reports, 8(1), 1478. https://doi.org/10.1038/s41598-018-19183-7
7. Loncle, C., Brunel, J. M., Vidal, N., Dherbomez, M., & Letourneux, Y. (2004). Synthesis and antifungal activity of cholesterol-hydrazone derivatives. European Journal of Medicinal Chemistry, 39(12), 1067-1071. https://doi.org/10.1016/j.ejmech.2004.07.005
8. Niu, W., Zheng, Y., Li, Y., Du, L., & Liu, W. (2021). Photochemical microfluidic synthesis of vitamin D₃ by improved light sources with photoluminescent substrates. Chinese Journal of Chemical Engineering, 29, 204-211. https://doi.org/10.1016/j.cjche.2020.07.016
9. Parish, E. J., & Wei, T.-Y. (1987). Allylic Oxidation of A 5-Steroids with Pyridinium Chlorochromate (PCC) and Pyridinium Dichromate (PDC). Synthetic Communications, 17(10), 1227-1233. https://doi.org/10.1080/00397918708063974
10. Yablonskaya, E. V., & Segal, M. G. (1973). Synthesis of 7-dehydrocholesterol acetate. Chemistry of Natural Compounds, 9(6), 708-709. https://doi.org/10.1007/BF00565791
11. Zhao, Q., Ji, L., Qian, G.-P., Liu, J.-G., Wang, Z.-Q., Yu, W.-F., & Chen, X.-Z. (2014). Investigation on the synthesis of 25-hydroxycholesterol. Steroids, 85,1-5. https://doi.org/10.1016/j.steroids.2014.02.002
12. Zmijewski, M. A., Li, W., Zjawiony, J. K., Sweatman, T. W., Chen, J., Miller, D. D., & Slominski, A. T. (2008). Synthesis and photo-conversion of androsta- and pregna-5,7-dienes to vitamin D₃-like derivatives. Photochemical & Photobiological Sciences, 7(12), 1570. https://doi.org/10.1039/b809005j

What is claimed is:

1. A process for making Vitamin D₃ from cholesterol derived from the processing of fish oil raw materials; wherein said cholesterol and Vitamin D₃ are characterized by the following structural formulas 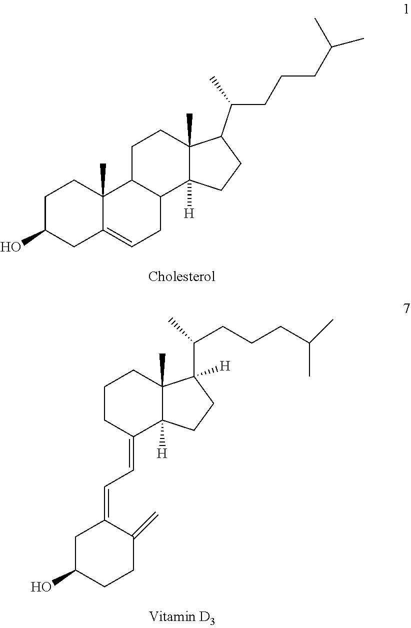 [SMILES: CC(C)CCC[C@@H](C)C1CC[C@H]2C3CC=C4C[C@@H](O)CC[C@]4(C)C3CC[C@]12C ; C=C1CC[C@@H](O)C/C1=C/C=C1\CCC[C@]2(C)[C@@H]([C@H](C)CCCC(C)C)CC[C@@H]12]
and wherein said process comprises the conversion of compound (1) to compound (7) using sequentially the following reaction steps (a) through (f):

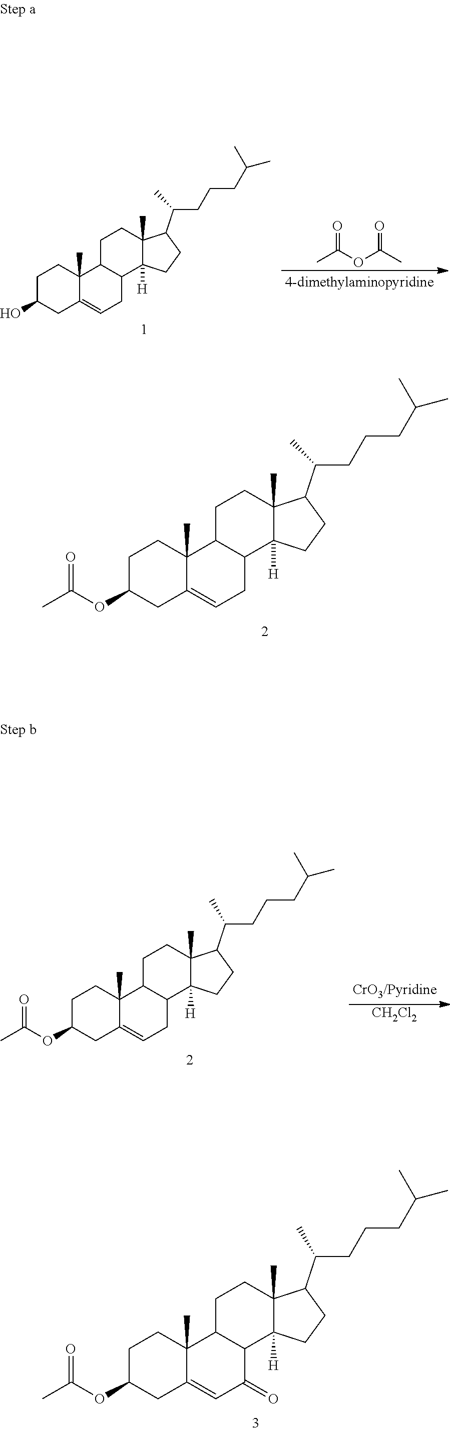

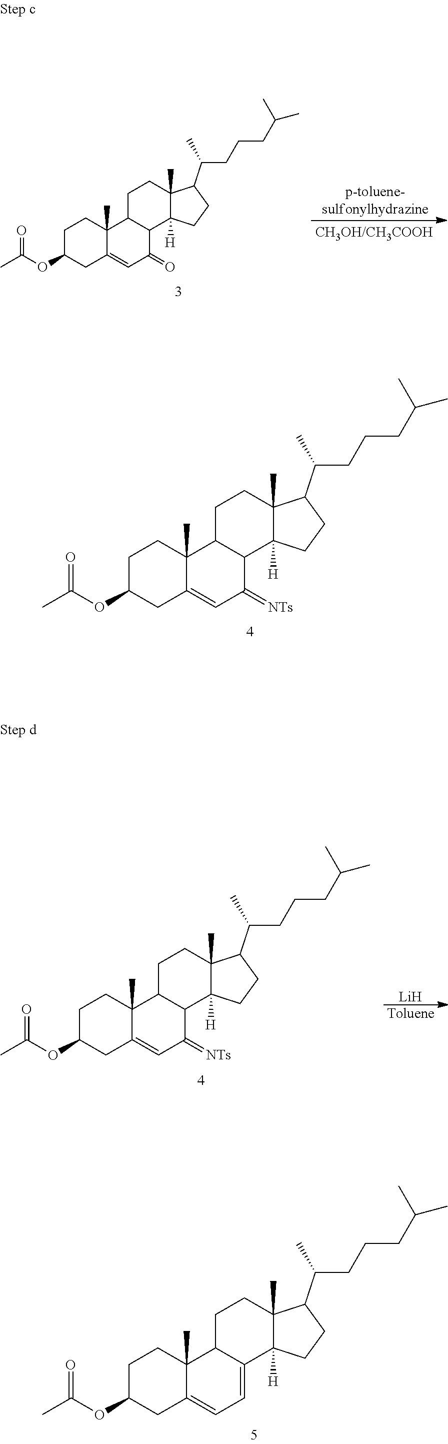

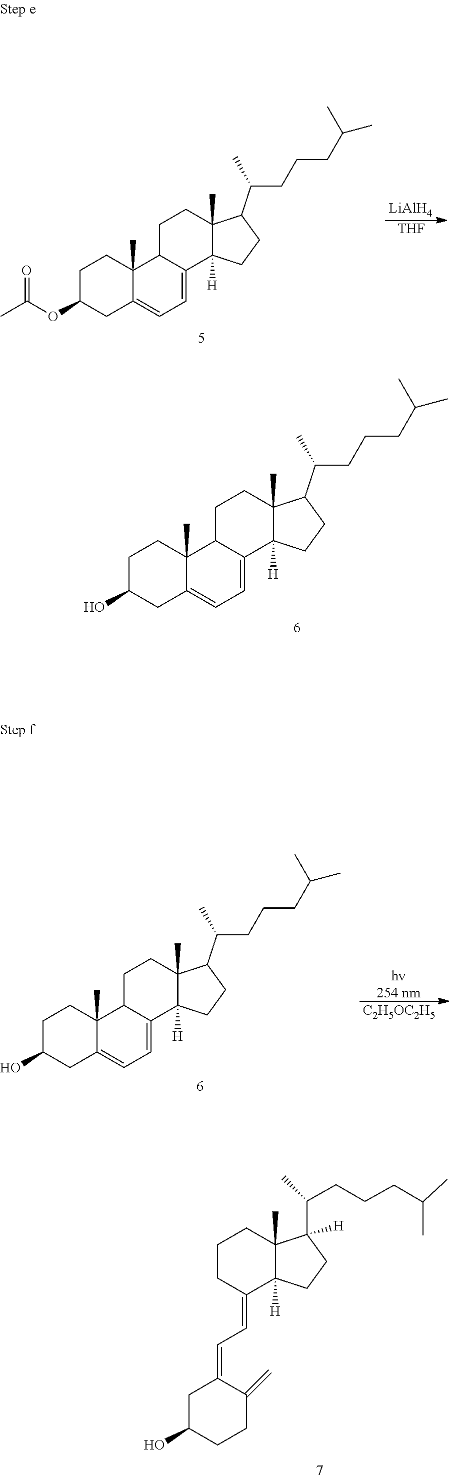

2. The method of claim 1, wherein said step (a) is conducted in a solvent selected from the group consisting of benzene, ethyl acetate, chloroform, methylene chloride, acetone, diethyl ether, hexane and cyclohexane.

3. The method of claim 2, wherein said solvent is hexane.

4. The method of claim 2, wherein said solvent is cyclohexane.

5. The method of claim 1, wherein said diethylether solvent used in step (f) is replaced by a solvent selected from the group consisting of methanol, ethanol, isopropanol, diisopropylether, tetrahydrofuran (THF), ethylformate, ethylacetate, acetonitrile, methylenechloride, ethylenedi-chloride, chloroform, benzene and toluene.

6. The method of claim 1, wherein the reaction time for step (b) is 20 hours.

7. The method of claim 1, wherein the reaction in (step c) is conducted under reflux conditions.

8. The method of claim 7, wherein said reflux is conducted for 7-8 hours.

9. The method of claim 1, wherein the reaction in (step d) is conducted under reflux conditions for 5 hours.

10. The method of claim 1, wherein the reaction in (step e) is first stirred for one hour under ice bath conditions and then stirred at room temperature for two hours.

11. The method of claim 1, wherein the reaction in (step f) is conducted at a temperature of 65° C., for 15 minutes.

12. A process for making Vitamin D₃ from cholesterol derived from the processing of fish oil raw materials, wherein said cholesterol and Vitamin D₃ are characterized by the following structural formulas 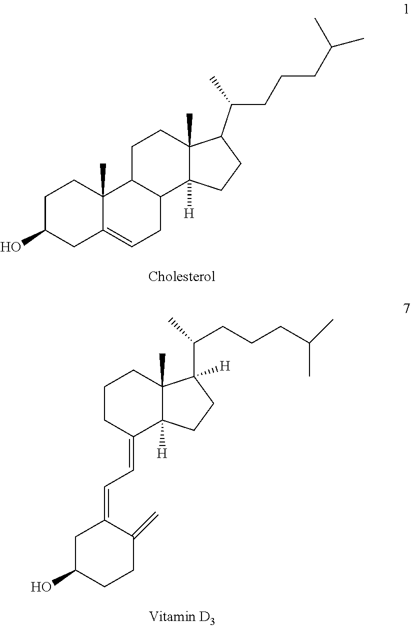 [SMILES: CC(C)CCC[C@@H](C)C1CC[C@H]2C3CC=C4C[C@@H](O)CC[C@]4(C)C3CC[C@]12C ; C=C1CC[C@@H](O)C/C1=C/C=C1\CCC[C@]2(C)[C@@H]([C@H](C)CCCC(C)C)CC[C@@H]12]
and wherein said process comprises the conversion of compound (1) to compound (7) using sequentially the following reaction steps (a) through (d):

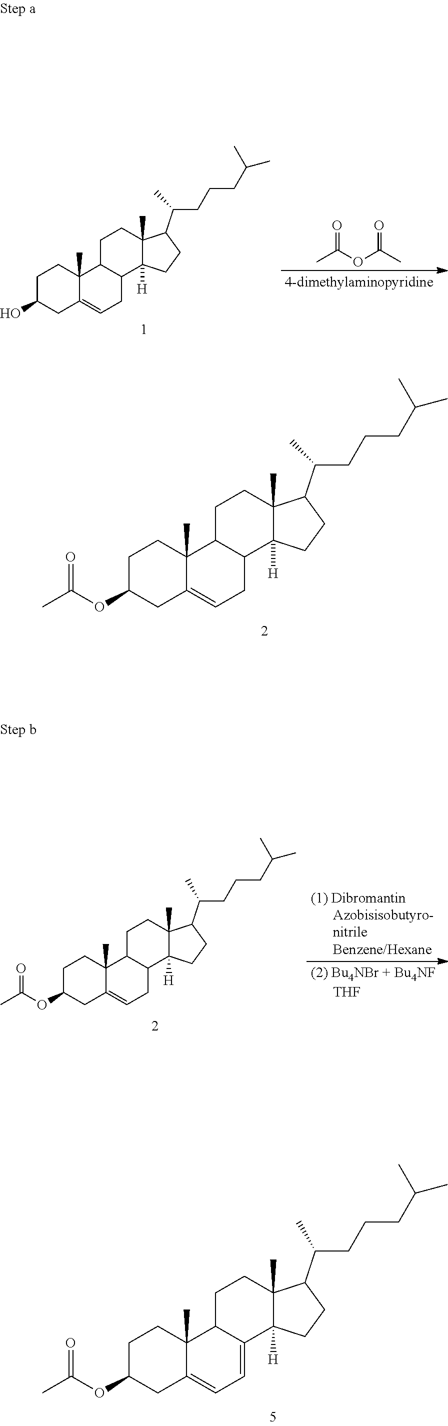

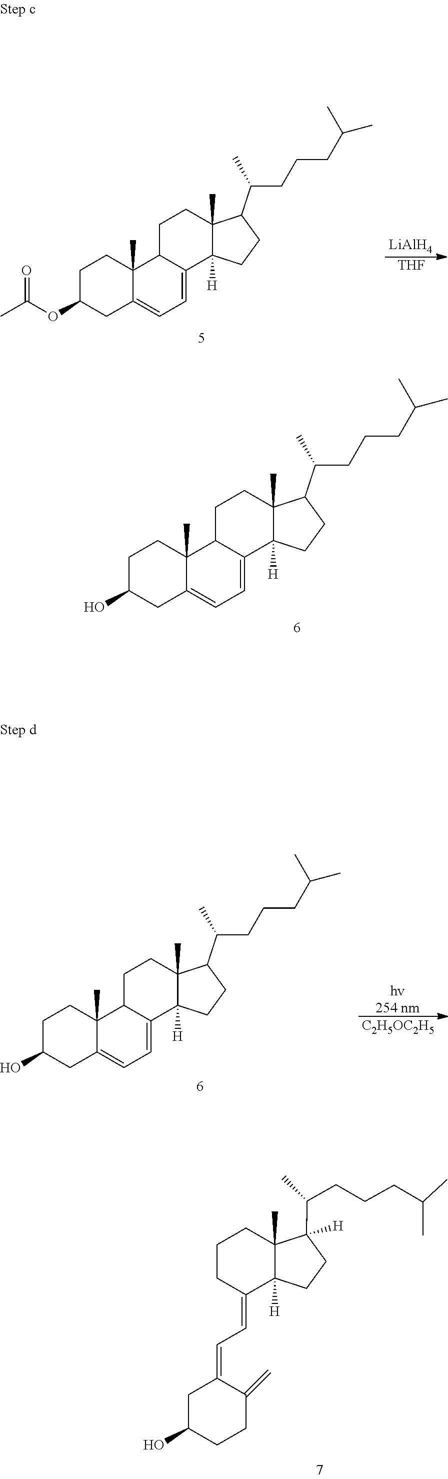

13. The method of claim 12, wherein said step (a) is conducted in a solvent selected from the group consisting of benzene, ethyl acetate, chloroform, methylene chloride, acetone, diethyl ether, hexane and cyclohexane.

14. The method of claim 13, wherein said solvent is hexane.

15. The method of claim 13, wherein said solvent is cyclohexane.

16. The method of claim 12, wherein step (1) of step (b) is conducted under reflux conditions for 10 minutes.

17. The method of claim 12, wherein in step (2) of step (b) the reaction with tetrabutylammonium bromide is conducted for 75 minutes.

18. The method of claim 12, wherein in step (2) of step (b) the reaction with tetrabutylammonium fluoride is conducted for 50 minutes.

19. The method of claim 12, wherein the reaction in (step d) is conducted at a temperature of 65° C., for 15 minutes.

20. The method of claim 12, wherein said diethylether solvent used in step (d) is replaced by a solvent selected from the group consisting of methanol, ethanol, isopropanol, diisopropylether, tetrahydrofuran (THF), ethylformate, ethylacetate, acetonitrile, methylenechloride, ethylenedi-chloride, chloroform, benzene and toluene.
